(19) United States
(12) Patent Application Publication
(10) Pub. No.: US 2024/0383971 A1
(43) Pub. Date: Nov. 21, 2024

(54) Anti-DFS70 Autoantibodies and methods of attenuating formation of nets

(71) Applicant: McMaster University, Hamilton (CA)

(72) Inventor: Manali Mukherjee, Hamilton (CA)

(21) Appl. No.: 18/199,248

(22) Filed: May 18, 2023

(51) Int. Cl. C07K 16/18; A61P 37/02

(52) U.S. Cl. CPC C07K 16/18; A61P 37/02

(57) ABSTRACT

A method of attenuating the formation of neutrophil extracellular traps (NETs) in a mammal is provided comprising administering to the mammal a therapeutically effective amount of DFS70 autoantibody, a functionally equivalent variant thereof or nucleic acid encoding DFS70 antibody.

### FIELD OF THE INVENTION

The present invention generally relates to autoantibodies, and in particular, to anti-DFS70 autoantibodies having therapeutic effects.

### BACKGROUND

Neutrophils comprise the largest population of myeloid leukocytes and function as the “first line” of defense against pathogens. NETosis is one neutrophilic antimicrobial mechanism in which they release modified chromatin studded with bactericidal proteins which can “trap” pathogens and confine their systemic spread. The formation of these neutrophil extracellular traps (NETs) can be triggered through various mechanisms including pattern recognition receptor activation (e.g. via lipopolysaccharides), FcR engagement (e.g. autoantibodies), and activated platelets.

Excessive and/or dysregulated NET formation has been suggested to mediate disease pathology in infections, inflammation, and autoimmunity. They have been shown to exacerbate certain viral infections (e.g. influenza, rhinovirus), in which the increase in NETosis markers such as myeloperoxidase-DNA and cell-free double stranded DNA (dsDNA) have been linked to worsened outcomes. Further, increased levels of NETs have been associated with acute respiratory distress syndrome (ARDS), requiring ventilatory support, oxygen supplementation, and intensive care. Indeed, many molecular components of NETs including dsDNA, histones, and granule proteins can function as autoantigens to trigger autoantibody production and the development of autoimmune diseases (e.g. systemic lupus erythematosus, rheumatoid arthritis). In turn, autoantibodies have the ability to induce NETosis, creating a positive feedback loop worsened by the persistent inflammation seen in autoimmune disorders.

The network structure of NETs can provide a scaffold for the deposition of erythrocytes, platelets, and other molecules conducive to subsequent thrombosis formation. The overactivation of the coagulation pathway can lead to vascular obstruction, resulting in dysregulated tissue blood supply. NETs have been observed ubiquitously in human arteriovenous thrombosis and deep vein thrombosis (DVT) models. In addition, the risk of thrombosis in severe COVID-19 has been well documented and is postulated to be exacerbated by increased NETosis in severe COVID patients.

### SUMMARY

It has now been determined that autoantibodies to DFS70 have both therapeutic and prognostic utilities which are applicable to the treatment of pathologies associated with NETs.

Thus, in one aspect, a method of attenuating the formation of neutrophil extracellular traps (NETs) in a mammal is provided comprising administering to the mammal a therapeutically effective amount of DFS70 autoantibody.

In another aspect, a method of attenuating platelet aggregation in a mammal is provided comprising administering to the mammal a therapeutically effective amount of DFS70 autoantibody.

In another aspect, a method of treating inflammation is provided comprising administering to a mammal in need a therapeutically effective amount of DFS70 autoantibody.

These and other aspects of the invention are described in detail by reference to the following figures.

### BRIEF DESCRIPTION OF THE FIGURES

FIG. 1 illustrates a detailed schematic showing in vitro experimental lay-out of inducing NETosis through various stimuli: 1. phorbol myristate acetate (PMA), 2. calcium ionophore A23187, 3. 10 μg immunoprecipitated immunoglobulins from antinuclear antibody positive serum.

FIG. 2 illustrates a step by step breakdown of anti-DFS70 antibody purification including: (A) immunoprecipitating immunoglobulins (IP-Ig) from healthy serum with detectable anti-DFS70 levels (measured with the antinuclear line immunoassay (ANA LIA) strips), and utilizing immunomagnetic separation and DFS70/LEDGF recombinant antigen (see online methods). (B) The resulting two immunoglobulin (Ig) fractions are with and without DFS70 antibodies, confirmed by (C) ANA LIA strips.

FIG. 3 illustrates that DFS70 autoantibodies act by inhibiting neutrophil NETosis. Scanning electron microscopy (SEM) images show that (A) NETs induced from DFS70-ANA+ patient serum in healthy neutrophils can be (B) attenuated in the presence of IP-Ig from DFS70+ IP from ANA+ serum. In an identical set of conditions set up in parallel, platelet-rich plasma from the same healthy donor were added 6 hrs post-IP-Ig (post-NETosis) to assess platelet aggregation events (D-E). All images were analyzed and quantified (C, F). Each data-point is an image analysis of a high-power field scan of the respective in vitro conditions. For individual DFS70+ sera testing from the respective “sub-groups” and DFS70 autoreactivities were re-tested on ANA strips. Care was taken not to repeat patient sera between the technical repeats/days of experiments. Each individual data point are MATLAB quantified data plotted from individual images of the experimental conditions. Representative images are given. Bars indicate magnification. Kruskal-Wallis with multiple comparison test.

FIG. 4 illustrates that DFS70 autoantibodies act by inhibiting neutrophil NETosis triggered by various stimuli. Scanning electron microscopy (SEM) images of (A) healthy donor neutrophils (pooled from n=4 donors) stimulated to NETosis through (B) A23187 and (C) PMA. (D) DFS70 negative IP-Ig from an ANA positive (ANA+) ARDS-ICU patient can also stimulate NETosis in healthy neutrophils, but is attenuated by additional treatment of DFS70 positive (DFS70+) IP-Ig from ANA+ patient serum. DFS70+ IP-Ig also attenuates NETosis induced by (E) A23187 and (F) PMA. (G) Heat inactivated DFS70+ IP-Ig does not attenuate NETosis. (H-I) DFS70+ IP-Ig from serum of healthy donors also attenuate NETosis induced by A23187 and PMA.

FIG. 5 illustrates an amino acid sequence of human DFS70 protein.

### DETAILED DESCRIPTION OF EMBODIMENTS OF THE INVENTION

A method to attenuate the formation of neutrophil extracellular traps (NETs) in a mammal is provided comprising administering to the mammal a therapeutically effective amount of a DFS70 autoantibody.

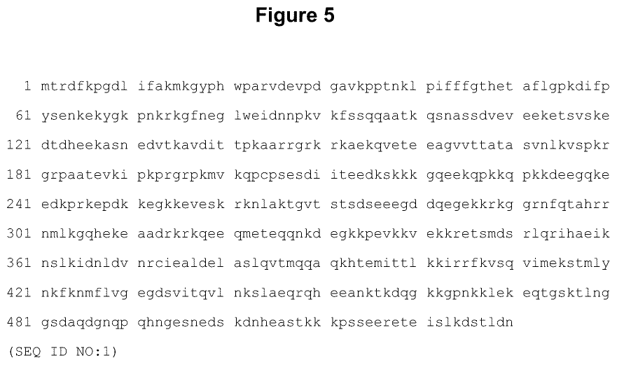

DFS70 (Dense Fine Speckled, 70 kDa molecular weight) autoantibody, also referred to herein as anti-DFS70 antibody, encompasses a sub-group of anti-nuclear IgG antibodies (ANA) against a 70 kDa antigen that gives rise to a fine dense speckled pattern (DFS) by indirect immunofluorescence. DFS70 is a chromatin-associated protein designated as dense fine speckled protein of 70 kD (DFS70), also known as lens epithelium-derived growth factor protein of 75 kD (LEDGF/p75) and PC4 and SFRS1 Interacting protein 1 (PSIP1). This multi-functional protein has been determined to have roles in the formation of transcription complexes in active chromatin, transcriptional activation of specific genes, regulation of mRNA splicing, DNA repair, and cellular survival against stress. As used herein, DFS70 refers to the mammalian protein, including human and non-human mammals, as well as functionally equivalent variants of the protein such as isoforms thereof that retain the antigenic properties thereof. DFS70 protein sequences are known and include, for example, the amino acid sequence of FIG. 5 (NCBI Reference Sequence: NP_001121689.1) and the sequence of NCBI Reference Sequence: NP_150091.2.

The term “functionally equivalent” refers to naturally or non-naturally occurring variants of the DFS70 autoantibody that retain the biological activity of the DFS70 autoantibody, e.g. to attenuate the formation of neutrophil extracellular traps (NETs) and/or platelet aggregation. In one embodiment, the functionally equivalent variant is an immunogenic variant of DFS70. The variant need not exhibit identical activity to endogenous DFS70 autoantibody, but will exhibit sufficient activity to render it useful to treat acute inflammation, e.g. at least about 25% of the biological activity of DFS70 autoantibody, and preferably at least about 50% or greater of the biological activity of DFS70 autoantibody. Such functionally equivalent variants may result naturally from alternative splicing during transcription or from genetic coding differences and may retain significant sequence homology with wild-type DFS70 autoantibody, e.g. at least about 70% sequence homology, preferably at least about 80% sequence homology, and more preferably at least about 90% or greater sequence homology. Such variants can readily be identified using established cloning techniques employing primers derived from DFS70 autoantibody. Additionally, such modifications may result from non-naturally occurring synthetic alterations made to DFS70 autoantibody to render functionally equivalent variants which may have more desirable characteristics for use in a therapeutic sense, for example, increased activity or stability. Non-naturally occurring variants of DFS70 autoantibody include analogues, fragments and derivatives thereof.

A functionally equivalent analogue of DFS70 autoantibody in accordance with the present invention may incorporate one or more amino acid substitutions, including additions and/or deletions. Amino acid additions or deletions include both terminal and internal additions or deletions to yield a functionally equivalent peptide. Examples of suitable amino acid substitutions include those made at positions within the protein that are not closely linked to activity, as well as conservative amino acid substitutions since such substitutions are less likely to adversely affect function. Examples of conservative substitutions include the substitution of a non-polar (hydrophobic) residue such as alanine, isoleucine, valine, leucine or methionine with another non-polar (hydrophobic) residue; the substitution of a polar (hydrophilic) residue with another such as between arginine and lysine, between glutamine and asparagine, between glutamine and glutamic acid, between asparagine and aspartic acid, and between glycine and serine; the substitution of a basic residue such as lysine, arginine or histidine with another basic residue; or the substitution of an acidic residue, such as aspartic acid or glutamic acid with another acidic residue.

A functionally equivalent fragment in accordance with the present invention comprises a portion of the DFS70 autoantibody sequence which maintains the function of intact DFS70 autoantibody. In one embodiment, the functionally equivalent fragment is an immunogenic fragment of DFS70. Such biologically active fragments of DFS70 autoantibody can readily be identified using assays useful to evaluate the activity of selected DFS70 autoantibody fragments to attenuate NETs and/or platelet aggregation, such as those herein described.

A functionally equivalent derivative of DFS70 autoantibody in accordance with the present invention is DFS70 autoantibody, or an analogue or fragment thereof, in which one or more of the amino acid residues therein is chemically derivatized. The amino acids may be derivatized at the amino or carboxy groups, or alternatively, at the side “R” groups thereof. Derivatization of amino acids within the peptide may yield a peptide having more desirable characteristics for use as a therapeutic such as increased stability or enhanced activity. Such derivatized molecules include for example, those molecules in which free amino groups have been derivatized to form, for example, amine hydrochlorides, p-toluene sulfonyl groups, carbobenzoxy groups, t-butyloxycarbonyl groups, chloroacetyl groups or formyl groups. Free carboxyl groups may be derivatized to form, for example, salts, methyl and ethyl esters or other types of esters or hydrazides. Free hydroxyl groups may be derivatized to form, for example, O-acyl or O-alkyl derivatives. The imidazole nitrogen of histidine may be derivatized to form N-im-benzylhistidine. Also included as derivatives are those peptides which contain one or more naturally occurring amino acid derivatives of the twenty standard amino acids, for example: 4-hydroxyproline may be substituted for proline; 5-hydroxylysine may be substituted for lysine; 3-methylhistidine may be substituted for histidine; homoserine may be substituted for serine; and ornithine may be substituted for lysine. Terminal derivatization of the protein to protect against chemical or enzymatic degradation is also encompassed including acetylation at the N-terminus and amidation at the C-terminus of the peptide.

DFS70 autoantibody, and functionally equivalent variants thereof, may be made using standard, well-established solid-phase peptide synthesis methods (SPPS). Two methods of solid phase peptide synthesis include the BOC and FMOC methods. DFS70 autoantibody and variants thereof may also be made using any one of a number of suitable techniques based on recombinant technology. It will be appreciated that such techniques are well-established by those skilled in the art, and involve the expression of DFS70 autoantibody-encoding nucleic acid in a genetically engineered host cell. Nucleic acid encoding DFS70 autoantibody may be synthesized de novo by automated techniques also well-known in the art given that the protein and nucleic acid sequences are known.

DFS70 autoantibody-encoding nucleic acid molecules or oligonucleotides may also be used to increase plasma DFS70 autoantibody levels in a mammal. In this regard, “DFS70 autoantibody-encoding nucleic acid” is used herein to encompass mammalian DFS70 autoantibody-encoding nucleic acid, including human and non-human forms, and functionally equivalent forms thereof (e.g. that encode functionally equivalent DFS70 autoantibody, or nucleic acids which differ due to degeneracy of the genetic code).

The term “oligonucleotide” refers to an oligomer or polymer of nucleotide or nucleoside monomers consisting of naturally occurring bases, sugars, and intersugar (backbone) linkages. The term also includes modified or substituted oligonucleotides comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells), or two or more oligonucleotides of the invention may be joined to form a chimeric oligonucleotide. Other oligonucleotides of the invention may contain modified phosphorous, oxygen heteroatoms in the phosphate backbone, short chain alkyl or cycloalkyl intersugar linages or short chain heteroatomic or heterocyclic intersugar linkages. For example, oligonucleotides may contain phosphorothioates, phosphotriesters, methyl phosphonates, and phosphorodithioates. Oligonucleotides of the invention may also comprise nucleotide analogs such as peptide nucleic acid (PNA) in which the deoxyribose (or ribose) phosphate backbone in the DNA (or RNA), is replaced with a polymide backbone similar to that found in peptides. Other oligonucleotide analogues may contain nucleotides containing polymer backbones, cyclic backbones, or acyclic backbones, e.g. morpholino backbone structures.

Such oligonucleotide molecules are readily synthesized using procedures known in the art based on the available sequence information. For example, oligonucleotides may be chemically synthesized using naturally occurring nucleotides or modified nucleotides as described above designed to increase the biological stability of the molecules or to increase the physical stability of the duplex formed with mRNA or the native gene, e.g. phosphorothioate derivatives and acridine substituted nucleotides. Selected oligonucleotides may also be produced biologically using recombinant technology in which an expression vector, e.g. plasmid, phagemid or attenuated virus, is introduced into cells in which the oligonucleotide is produced under the control of a regulatory region.

As one of skill in the art will appreciate, antibodies to the target DFS70 protein may also be raised using techniques conventional in the art. For example, antibodies may be made by injecting a non-human host animal, e.g. a mouse or rabbit, with the antigen (target protein or immunogenic fragment thereof), and then isolating antibody from a biological sample taken from the host animal.

Once prepared and suitably purified, DFS70 autoantibody, DFS70 autoantibody-encoding oligonucleotides, or functionally equivalent variants thereof, may be utilized in accordance with the invention to treat inflammation. In this regard, increasing the expression of DFS70 autoantibody in a mammal, by administration of DFS70 autoantibody or by administration of DFS70 autoantibody-encoding nucleic acid, results in DFS70 autoantibody expression or over-expression in the mammal. While not wishing to be bound by any particular mode of action, upregulation of DFS70 autoantibody appears to limit autoimmune pathology by arresting NETosis and subsequent platelet aggregation.

Thus, the present method is useful generally to treat pathologies associated with the formation of excess neutrophil extracellular traps (NETs) or NETosis including, but not limited to, inflammation, autoimmune and autoinflammatory diseases such as psoriasis, systemic lupus erythematosus (SLE), rheumatoid arthritis (RA), type 1 diabetes mellitus (T1DM), small vessel vasculitis (SVV), metabolic disease, inflammatory bowel disease, type2 diabetes and obesity, acute respiratory distress syndrome (ARDs), COPD, thrombotic events, sepsis and disease resulting from a pathogenic infection, such as COVID.

DFS70 autoantibody or nucleic acid encoding DFS70 autoantibody may be administered either alone or in combination with at least one pharmaceutically acceptable adjuvant, for use in treatments in accordance with embodiments of the invention. The expression “pharmaceutically acceptable” means acceptable for use in the pharmaceutical and veterinary arts, i.e. not being unacceptably toxic or otherwise unsuitable. Examples of pharmaceutically acceptable adjuvants are those used conventionally with peptide- or nucleic acid-based drugs, such as diluents, excipients and the like. Reference may be made to “Remington's: The Science and Practice of Pharmacy”, 21st Ed., Lippincott Williams & Wilkins, 2005, for guidance on drug formulations generally.

The selection of adjuvant depends on the intended mode of administration of the composition. In one embodiment of the invention, the compounds are formulated for administration by infusion, or by injection either subcutaneously or intravenously, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. Compositions for oral administration via tablet, capsule or suspension are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, anti-oxidants, preservatives, colouring agents and flavouring agents may also be present. Formulations for administration intranasally, or by inhalation, may also be prepared in saline or other suitable buffer and/or propellant adjuvants, to be nebulized to form a liquid aerosol for inhalation by mouth or nasally. Other adjuvants may also be added to the composition regardless of how it is to be administered, for example, anti-microbial agents may be added to the composition to prevent microbial growth over prolonged storage periods.

Therapeutic DFS70 autoantibody-encoding oligonucleotides may be directly administered in vivo formulated, for example, in saline or an appropriate buffer. Alternatively, the oligonucleotides may be introduced into tissues or cells using techniques in the art including vectors (retroviral vectors, adenoviral vectors and DNA virus vectors) or physical techniques such as microinjection, and then administered in vivo. Administration of such cells may be achieved, for example, by encapsulated cell biodelivery.

For use to attenuate NETS, a therapeutically effective amount of DFS70 autoantibody or nucleic acid encoding DFS70 autoantibody is administered to a mammal. As used herein, the term “mammal” is meant to encompass, without limitation, humans, domestic animals such as dogs, cats, horses, cattle, swine, sheep, goats and the like, as well as non-domesticated animals. The term “therapeutically effective amount” is an amount of the DFS70 autoantibody or nucleic acid encoding DFS70 autoantibody required to attenuate NETs and/or platelet aggregation, while not exceeding an amount which may cause significant adverse effects. Dosages of DFS70 autoantibody, functionally equivalent variants thereof, or nucleic acid encoding it, that are therapeutically effective will vary on many factors including the nature of the condition to be treated as well as the particular individual being treated. In embodiments, dosages of DFS70 autoantibody in the range of about 1-500 mg, for example 10-200 mg, or a dosage of nucleic acid encoding DFS70 autoantibody that expresses about 1-500 mg of DFS70. The dosage may be a single dosage, a total dosage administered over a period of time such as 2 or more days, or a daily dosage administered over a period of time, e.g. 2 or more days.

In the present treatment, DFS70 autoantibody or nucleic acid may be administered by any route suitable to increase the plasma levels thereof. Examples of suitable administrable routes include, but are not limited to, oral, subcutaneous, intravenous, intraperitoneal, intranasal, enteral, topical, sublingual, intramuscular, intra-arterial, intramedullary, intrathecal, inhalation, ocular, transdermal, vaginal or rectal means. Depending on the route of administration, the protein or nucleic acid may be coated or encased in a protective material to prevent undesirable degradation thereof by enzymes, acids or by other conditions that may affect the therapeutic activity thereof.

In an embodiment, administration of DFS70 autoantibody or nucleic acid is conducted by inhalation, including intranasally, to target the lung.

In one embodiment, DFS70 autoantibody or nucleic acid may be administered alone, in combination with, (either together, simultaneously with or administered at different times) at least one other therapeutic compound such as a compound effective to treat a target pathology. For example, DFS70 autoantibody may be used in combination with NET-inhibiting pharmacologic interventions such as dexamethasone, JAK2 inhibitors (such as barcitinib), disulfiram, rituximab, PKC inhibitor treatments, PAD4 inhibitors (e.g. Cl-amidine, hydroxychloroquine), antibiotics (such as azithromycin, gentamicin), DNase therapy, and calcineurin inhibitor (e.g. cyclosporine A). DFS70 autoantibodies may also be administered with a therapeutic that targets non-NETosis aspects of the condition to be treated, and/or anti-inflammatory medications.

Embodiments of the invention are described by reference to the following specific examples which are not to be construed as limiting.

### Example

The following work was done to exemplify embodiments of the invention.

### Methods

Sample Procurement & Processing-Peripheral blood was drawn in anti-coagulant-free vacutainers for serum processing. The immunoglobulins from serum were immunoprecipitated as previously described (Mukherjee et al. Allergy Asthma Clin Immunol 13, 2 (2017). The eluted immunoprecipitated immunoglobulins (IP-Igs) were utilized for downstream assays, described below.

Detection and Quantification of Anti Extractable Nuclear Antibodies in Serum & ETA-Antibodies to nuclear antigens in serum and ETA samples were detected using anti/extractable nuclear antibody line immunoassay (IMTEC-ANA-LIA-MAXX, Human Diagnostics, Germany), as previously described (Mukherjee et al. Allergy and Clinical Immunology 141, 1269-1279 (2018)). This immunoassay measured autoantibodies for the following 18 anti/extractable nuclear antibodies (ANA/ENA): dsDNA, nucleosome, histone, SmD1, PCNA, RO/(RPP), SS-A/Ro 60, SS-A/Ro 52, SS-B/La, CENP-B, Scl70, U1-snRNP, AMA M2, Jo-1, PM-Scl, Mi-2, Ku, and DFS70. Disease modifying titers of 1:100 and 1:8 was used for serum and ETA respectively, and samples were incubated overnight at 4° C. Each strip was scanned (ChemiDoc MP Imaging System, Bio-Rad, CA, USA) and converted into 8-bit grayscale images and inverted with ImageJ analysis software (National Institute of Health, MD, USA). A quantitative value was derived for each visible band and normalized to the cut-off control band to provide a mean quantitative value (MQV) with values >1.0 indicating positive reactivity. The ANA reactivities were validated using indirect immunofluorescence (IIF) on human epithelial (HEp-2) cells (Euroimmune, Lübeck, Germany), the gold standard screening method for ANA detection. In brief, the HEp-2 cell substrates were incubated with patient sera at 1:100 dilution and visualized for anti-cell (AC) patterns from the International Consensus on ANA Patterns (ICAP) to verify reactivities on the line immunoassay strip. Given the unique observation of high DFS70 reactivity, we further validated the LIA with the HEp-2 AC-2 patterns (agreement Cohen's Kappa 0.852) on 36 samples with adequate sera volume.

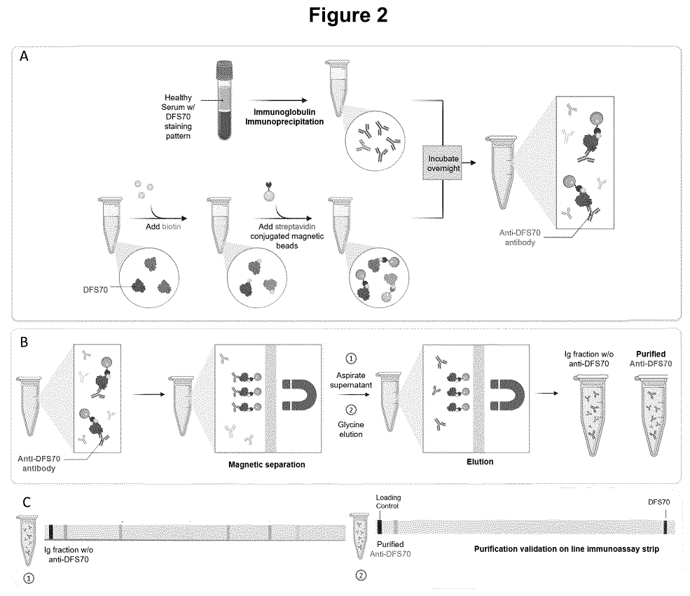

Anti-DES70 Antibody Purification-LEDGF/DFS70 recombinant protein (R&D Systems, MN, USA) was biotinylated (EZ-Link™ NHS-PEG4-Biotinylation Kit, Thermo Fisher, MA, USA) and allowed to bind to paramagnetic streptavidin beads (Dynabeads™ M-280, Thermo Fisher) as per manufacturers' protocols (FIG. 2A). The protein-biotin-streptavidin complex was incubated overnight (4° C.) with immunoprecipitated-immunoglobulins (IP-Igs) from n=4 serum samples with high anti-DFS70 reactivity as per the ANA line immunoassay described above. The mixture was placed in a magnet (DynaMag™-2, Thermo Fisher) and the purified anti-DFS70 Ig fraction remained bound to the tube wall while the unbound Igs were collected in a separate tube. The bound Ig fraction was washed thoroughly with PBS and eluted from the DFS70 protein with glycine (pH 2.7) and neutralized with Tris buffer (FIG. 2B). Both the purified and unbound Ig fraction reactivities were assessed via line immunoassay (FIG. 2C) and Hep2 indirect immunofluorescence.

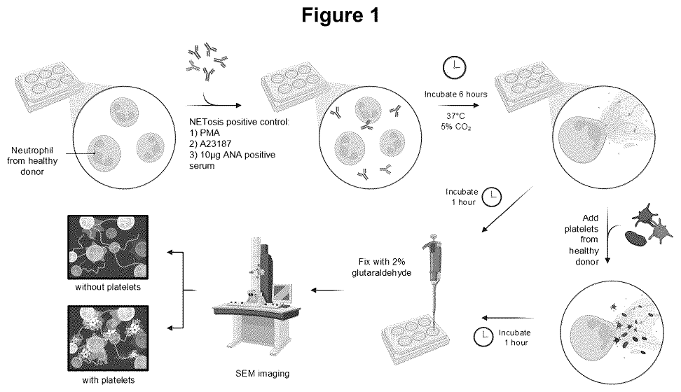

In Vitro NETosis Assay and Platelet Aggregation—Neutrophils were isolated from peripheral blood of healthy donors (no history of autoimmune disorders, COVID-negative on PCR/rapid antigen test or no history of exposure, no infection/symptoms in previous 4 weeks) using the MACSxpress neutrophil isolation kit as previously described (Son et al. Journal of Immunological Methods 449, 44-55 (2017)), and pooled post-isolation (n=4 donors) (FIG. 1). The purified population was resuspended in RPMI1640 at 106 cells/mL, seeded on poly-l-lysine-(100 μg/mL) and fibronectin-coated (2 μg/mL in PBS) coverslips, and subsequently treated with 10 μg IP-Ig from an ICU-admitted ANA+patient with no anti-DFS70 reactivity (>1.0 MQV as per ANA quantification described above) or purified anti-DFS70 antibodies and its eluted fraction (method described above) (FIG. 1). The above set up was maintained for all the technical repeats of this experiment reported. In addition, as positive controls for NETosis, Phorbol 12-myristate 13-acetate (PMA, 100 nM) and calcium ionophore A23187 (25 μM) were used, and 10 μg Chrompure IgG (Jackson Immune-Research, MO, USA) as non-specific control (FIG. 1). Pre-incubation with FcR block for 20 minutes prior to autoantibody positive serum treatment was done to observe NETosis inhibition. In parallel, dexamethasone (10 μM, Sigma Aldrich) and JAK2 inhibitor (1 nM, CAS 457081-03-7, Sigma Aldrich) were added to neutrophils treated for NETosis resolution. All conditions were incubated for 6 hrs at 37° C. and 5% CO₂. Platelet aggregation was assessed by adding platelet-rich plasma isolated from the same n=4 healthy donors to an identical set of conditions set up in parallel for an additional 1 hr. The samples were fixed with 4% paraformaldehyde and 2% glutaraldehyde for immunofluorescence and scanning electron microscopy (SEM), respectively (FIG. 1). Immunofluorescence with anti-histone staining was used to develop and validate the SEM NETosis protocols.

Scanning Electron Microscopy-Samples were fixed in 2% glutaraldehyde (2% v/v) in 0.1M sodium cacodylate buffer pH 7.4. The samples were rinsed 2× in buffer solution, post-fixed in 1% osmium tetroxide in 0.1M sodium cacodylate buffer for 1 hour and then dehydrated through a graded ethanol series (50%, 70%, 70%, 95%, 95%, 100%, 100%). The samples were kept immersed in 100% EtOH, placed into wire baskets and transferred to the chamber of a Leica EM CPD300 critical point dryer (Leica Mikrosysteme GmbH, Wien, Austria). The chamber was sealed and then flushed 12 times with liquid CO₂. The CO₂ filled chamber was heated to 35° C. and pressure was increased in the chamber to above 1100 psi so that CO₂ was changed from liquid phase to gaseous phase. The gas was vented slowly from the chamber until atmospheric pressure was reached and the samples were dehydrated without surface tension damage. The dried samples were mounted onto SEM stubs with double-sided carbon tape. The samples on stubs were then placed in the chamber of a Polaron Model E5100 sputter coater (Polaron Equipment Ltd., Watford, Hertfordshire) and approximately 20 nm of gold was deposited onto the stubs. The samples were viewed in a Tescan Vega II LSU scanning electron microscope (Tescan USA, PA) operating at 10 kV.

Image quantification-SEM images were processed using an automated algorithm developed in MATLAB 2016a (MathWorks, Natick, MA). The greyscale SEM images were binarized using a k-means clustering algorithm. Subsequently, the number of pixels in each yellow region was measured using a 2D pixel connectivity toolset for a 4-connected pixel neighborhood in MATLAB. To evaluate differences in the amount of neutrophil-derived extracellular traps (NETs) between images/conditions, the fraction of yellow pixels was calculated for each image. To evaluate the amount of platelet agglomeration in each image the number of connected regions larger than 2 cells was calculated as a fraction of total number of regions in each of the images that were larger than 5 pixels. The number of pixels in a single cell (i.e., 199) was calculated as the average number of pixels in 5 randomly selected cells.

Statistical analysis-GraphPad Prism software (Version 9, CA, USA) was used for statistical analyses and plotting. After testing for normality, two-sided Mann-Whitney and Kruskal-Wallis (Dunn's multiple comparisons test post hoc) non-parametric tests were performed to compare between 2 and >2 groups, respectively. Associations were determined by Spearman's rank correlation test, and categorical variable frequencies via Chi-squared analysis. Regression analyses (univariate and multivariate) generated predictive models using the R software (URL: https://www.stats.ox.ac.uk/pub/MASS4/). P<0.05 was considered to be significant.

### Results

Specific Autoantibodies Associated with Severe Inflammation Pathology and Mortality—Diverse circulating autoantibodies have been detected in patients with severe inflammatory disease, of which ANAs (>1:160 titer via Hep-2 immunofluorescence) were reported most commonly in up to 48% of the most severe subset. We used a validated rapid line immunoassay (LIA) that detects specific autoreactivities to 18 common ANA/ENAs (nuclear and extractable nuclear antigens) at 1:100 titer. The observed reactivities were further confirmed through the International Consensus for ANA patterns (ICAP, AC-patterns) (www.anapatterns.org,) using gold standard indirect immunofluorescence of HEp-2 cells.

Given the novel observation with DFS70 autoreactivity, we further validated the LIA with HEp-2 patterns (Cohen's Kappa: 0.852) on 36 samples with adequate sera volume. The AC-2 pattern produced by autoantibodies to DFS70 is due to a nuclear chromatin-associated protein of approximately 70 kDa. We further confirmed the reactivity through dot blots using commercially available recombinant DFS70 protein (R&D Systems, MN, USA).

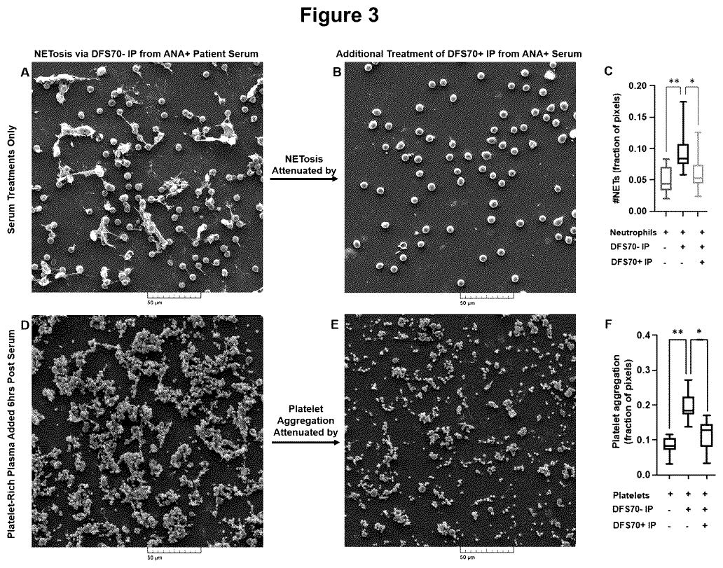

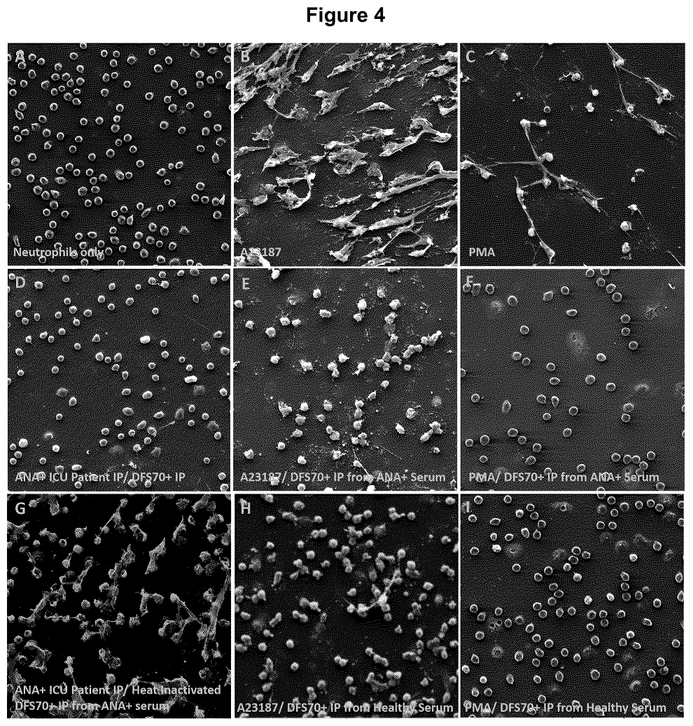

Autoantibody Triggered Neutrophil Extracellular Traps are Reduced in Patients with Anti-DES70 Antibodies—To assess autoantibody-mediated pathology, we used immunoprecipitated immunoglobulins (IP-Igs) from serum from an ICU-admitted ANA+ patient to induce NETosis in neutrophils isolated from healthy donors. Commercially available non-specific IgG was used as a negative control, and calcium ionophore (A23187) (FIG. 4B) and phorbol myristate acetate (PMA) (FIG. 4C) as positive controls to induce NETs. Given the reduced potential of sera with higher detectable titers of anti-DFS70 IgG to induce NETosis in vitro, we hypothesized that naturally occurring anti-DFS70 IgG limited autoimmune pathology by arresting NETosis and subsequent platelet aggregation. An earlier study reported purified autoantibodies from some patients activated neutrophils in vitro and potentiated thrombosis when injected into mice. In fact, increased NETs are known to promote platelet aggregation and thrombosis, and the latter has been assigned to be the main cause of COVID mortality. Therefore, we assessed the ability of IP-Igs from a patient with high autoantibody titers (but DFS70 negative) to induce NETs in vitro (FIG. 3A), followed by subsequent aggregation of platelets from healthy donors (FIG. 3D). In addition, we assessed the capability of sera with/without anti-DFS70 antibodies to attenuate IP-Ig-induced NETosis. Indeed, IP-Ig from sera where anti-DFS70 IgG was present significantly reduced NETs (FIG. 3B, 4D) induced by ANA+ serum IP (FIG. 4D), A23187 (FIG. 4E), and PMA (FIG. 4F). Fc receptor (FcR) block was used as a positive control for preventing NETosis. Finally, in every condition that attenuated NETs including the presence of anti-DFS70 IgG, the aggregation of platelets was significantly arrested (FIG. 3E).

Purified Anti-DFS70 IgG Arrests NETosis and Subsequent Thrombotic Events-We purified anti-DFS70 antibodies from patient and healthy sera using immunomagnetic separation protocols. The purity and autoreactivity were tested on the ANA LIA, dot blots, and HEp-2 indirect immunofluorescence staining. Similar in vitro experiments as previously highlighted were set up with neutrophils from healthy donors (n=4, pooled for each technical repeat) to assess the attenuation of autoantibody-induced (sourced from an ANA positive patient) NETosis and subsequent platelet aggregation. Purified anti-DFS70 antibodies extracted from patient and healthy sera significantly stopped autoantibody mediated NETosis in vitro and platelet aggregation compared to the “control” (eluted immunoglobulin fraction without anti-DFS70 IgG). In a similar set of conditions, purified anti-DFS70 IgG from sera of healthy individuals also attenuated NETosis events (FIG. 4H-I), indicating that the observed phenomenon induced by anti-DFS70 autoantibodies was not inflammation/disease-specific. The ability to arrest NETosis was reversed when the purified anti-DFS70 antibody was heat inactivated (FIG. 4G). In our study, the number of documented thrombotic adverse events were proportionally greater in the recruited patients without anti-DFS70 IgG compared to those in with detectable serum titer >1:100.

### Discussion

The association of autoantibodies, neutrophil extracellular traps, and cytokine storm with infection and inflammation severity is well documented. Consistent with a role for NETs in driving fatality, airway secretions from patients with more severe outcomes had higher detectable dsDNA. In addition, immunoprecipitated immunoglobulins (IP-Igs) from an ANA positive patient triggered increased NETosis in vitro with subsequent platelet aggregation compared to ANA negative sera/IP-Igs, establishing a possible mechanism for the observed increase in thrombotic events.

As set out herein, it was determined in the present study that the inability of the host to limit autoimmune-triggered NETs and thrombotic events is due to the absence of a naturally occurring autoantibody that targets a nuclear chromatin-associated protein of approximately 70 kDa, known as DFS70. We are the first to report that the presence of circulating anti-DFS70 IgG was significantly associated with a favorable outcome in critically ill patients, irrespective of other pathogenic autoantibodies or pre-existing comorbidities or therapeutic interventions. The protective role of these autoantibodies could be explained by their ability to limit systemic hyper-coagulopathy by arresting NETosis and subsequent platelet aggregation.

To our knowledge, we are the first to demonstrate naturally occurring autoantibodies to DFS70 attenuate NETosis and play a protective role in severe infection/inflammation. Further, purified DFS70 antibody from patients and healthy donors had comparable effect on arresting NETosis and provides insight that the protective mechanism is not inflammation/disease-specific. The clinical importance of arresting NETosis in pathology and reducing subsequent clotting mechanisms is evident from the successful clinical trials/outcomes using NET-inhibiting pharmacologic interventions such as dexamethasone or JAK2 inhibitor (barcitinib). The discovery of an endogenous autoantibody that can arrest NET formation to a comparable degree as dexamethasone and barcitinib is clinically and scientifically empirical, with implications for infection diseases and autoimmunity.

1. A method of attenuating the formation of neutrophil extracellular traps (NETs) in a mammal comprising administering to the mammal a therapeutically effective amount of DFS70 autoantibody, a functionally equivalent variant thereof or nucleic acid encoding DFS70 antibody.

2. The method of claim 1, wherein the DFS70 antibody is administered to the mammal.

3. The method of claim 1, wherein the mammal is a human.

4. The method of claim 1, wherein the mammal has an autoimmune disease, autoinflammatory disease, inflammation, sepsis, thrombolytic disease or a disease resulting from a pathogenic infection.

5. The method of claim 1, wherein the mammal has ARDS.

6. The method of claim 1, wherein a dosage in the range of about 1-500 mg of the DFS70 antibody is administered to the mammal.

7. The method of claim 1, wherein the DFS70 antibody, variant or nucleic acid is administered by a route suitable to increase the plasma levels thereof.

8. The method of claim 1, the DFS70 antibody, variant or nucleic acid is administered by inhalation.

9. The method of claim 1, wherein the DFS70 antibody, variant or nucleic acid is administered in combination with a different NET-inhibiting pharmacologic intervention.

10. The method of claim 1, to treat inflammation in the mammal.

11. A method of preventing platelet aggregation in a mammal comprising administering to the mammal a therapeutically effective amount of DFS70 autoantibody, a functionally equivalent variant thereof or nucleic acid encoding DFS70 antibody.
